(19) United States
(12) Patent Application Publication
(10) Pub. No.: US 2020/0188261 A1
(43) Pub. Date: Jun. 18, 2020

(54) TOPICAL COMPOSITIONS AND METHODS OF USING THE SAME

(71) Applicant: Novan, Inc., Morrisville, NC (US)

(72) Inventors: Nathan Stasko, Chapel Hill, NC (US); Ryan Doxey, Raleigh, NC (US)

(21) Appl. No.: 16/623,834

(22) PCT Filed: Jun. 19, 2018
(86) PCT No.: PCT/US2018/038188

§ 371 Date: Dec. 18, 2019

Related U.S. Application Data
(60) Provisional application No. 62/521,884, filed on Jun. 19, 2017.

(51) Int. Cl. A61K 8/44; A61Q 19/08; A61K 8/41; A61K 8/81; A61Q 19/10

(52) U.S. Cl. CPC A61K 8/44; A61Q 19/08; A61K 8/41; A61K 8/8152; A61K 2800/51; A61K 2800/244; A61K 2800/88; A61K 2800/48; A61Q 19/10

(57) ABSTRACT

The present invention relates generally to compositions and methods of using the same such as, for example, methods of treating the skin of a subject using a composition as described herein. The composition may comprise a diluent, a surfactant, a thickening agent, a humectant, a cooling agent, a chelating agent, optionally a pH adjustment agent, and optionally a preservative. The composition may further comprise L-arginine and/or L-citrulline and/or esters and/or derivatives thereof.

### RELATED APPLICATION INFORMATION

This application claims the benefit of U.S. Provisional Patent Application Ser. No. 62/521,884, filed Jun. 19, 2017, the disclosure of which is incorporated herein by reference in its entirety.

### FIELD

The present invention relates generally to compositions and methods of using the same.

### BACKGROUND

Skin may be cleaned and/or treated with various compositions. The present invention addresses previous shortcomings in the art by providing topical compositions and methods of using the topical compositions.

### SUMMARY

A first aspect of the present invention is directed to a composition comprising: a diluent, a surfactant, a thickening agent, a humectant, a cooling agent, a chelating agent, optionally a pH adjustment agent, and optionally a preservative. In some embodiments, the composition further comprises L-arginine and/or L-citrulline and/or esters and/or derivatives thereof. The composition may be cosmetically elegant. The composition may be a pharmaceutical composition.

A further aspect of the present invention is directed to a kit comprising a composition of the present invention.

Another aspect of the present invention is directed to a method of cleaning the skin of a subject, the method comprising applying a composition of the present invention to the skin of the subject. In some embodiments, provided is a method of treating a subject, the method comprising applying a composition of the present invention to the skin of the subject.

Another aspect of the present invention is directed to a method of treating acne vulgaris comprising topically applying a composition of the present invention to the skin of a subject. A therapeutically effective amount of the composition may be applied.

A further aspect of the present invention is directed to a method of reducing inflammatory and/or noninflammatory lesions in a subject comprising topically applying a composition of the present invention to the skin of the subject. A therapeutically effective amount of the composition may be applied. The method may reduce inflammatory and/or noninflammatory lesions by about 10% or greater over a defined period of time compared to a subject who did not apply a composition of the present invention over the same time period. The subject may see a reduction in inflammatory and/or noninflammatory lesions within 12 weeks or less, in some embodiments, within 8 weeks or less, and in further embodiments, within 4 weeks or less.

Another aspect of the present invention is directed to a method of reducing P. acnes counts in a subject comprising topically applying a composition of the present invention to the skin of the subject. A therapeutically effective amount of the composition may be applied. The method may reduce P. acnes counts by about 10% or greater over a defined period of time compared to a subject who did not apply a composition of the present invention over the same time period. The subject may see a reduction in P. acnes counts within 12 weeks or less, in some embodiments, within 8 weeks or less, and in further embodiments, within 4 weeks or less.

The foregoing and other aspects of the present invention will now be described in more detail with respect to other embodiments described herein. It should be appreciated that the invention can be embodied in different forms and should not be construed as limited to the embodiments set forth herein. Rather, these embodiments are provided so that this disclosure will be thorough and complete, and will fully convey the scope of the invention to those skilled in the art.

### DETAILED DESCRIPTION

The present invention will now be described more fully hereinafter. This invention may, however, be embodied in different forms and should not be construed as limited to the embodiments set forth herein. Rather, these embodiments are provided so that this disclosure will be thorough and complete, and will fully convey the scope of the invention to those skilled in the art.

The terminology used in the description of the invention herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the present application and relevant art and should not be interpreted in an idealized or overly formal sense unless expressly so defined herein. The terminology used in the description of the invention herein is for the purpose of describing particular embodiments only and is not intended to be limiting of the invention. All publications, patent applications, patents and other references mentioned herein are incorporated by reference in their entirety. In case of a conflict in terminology, the present specification is controlling.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a complex comprises components A, B and C, it is specifically intended that any of A, B or C, or a combination thereof, can be omitted and disclaimed.

As used herein, the transitional phrase “consisting essentially of” (and grammatical variants) is to be interpreted as encompassing the recited materials or steps “and those that do not materially affect the basic and novel characteristic(s)” of the claimed invention. See, In re Herz, 537 F.2d 549, 551-52, 190 U.S.P.Q. 461, 463 (CCPA 1976) (emphasis in the original); see also MPEP § 2111.03. Thus, the term “consisting essentially of” as used herein should not be interpreted as equivalent to “comprising.”

The term “about,” as used herein when referring to a measurable value, such as an amount or concentration and the like, is meant to refer to variations of up to ±20% of the specified value, such as, but not limited to, ±10%, ±5%, ±1%, ±0.5%, or even ±0.1% of the specified value, as well as the specified value. For example, “about X” where X is the measurable value, is meant to include X as well as variations of ±20%, ±10%, ±5%, ±1%, ±0.5%, or even ±0.1% of X. A range provided herein for a measureable value may include any other range and/or individual value therein.

According to some embodiments of the present invention, provided herein are topical compositions. A composition of the present invention may comprise a diluent, a surfactant, a thickening agent, a humectant, a pH adjustment agent, a cooling agent, a chelating agent, a preservative, L-arginine, L-citrulline, and/or an ester and/or derivative of L-arginine and/or L-citrulline. In some embodiments, the composition may be a foaming cleanser and/or may foam upon applying the composition to the skin of a subject.

One or more (e.g., 1, 2, 3, 4, 5, or more) diluent(s) may be present in the composition. Example diluents include, but are not limited to, water. In some embodiments, a diluent is present in an amount of about 35%, 36%, 37%, 38%, 39%, 40%, 41%, 42%, 43%, 44%, 45%, 46%, 47%, 48%, 49%, 50%, 51%, 52%, 53%, 54%, or 55% by weight of the composition. In some embodiments, a diluent is present in an amount of about 35%, 36%, 37%, 38%, 39%, or 40% to about 41%, 42%, 43%, 44%, 45%, 46%, 47%, 48%, 49%, 50%, 51%, 52%, 53%, 54%, or 55% by weight of the composition. In some embodiments, the total amount of the one or more diluent(s) in the composition is about 35%, 36%, 37%, 38%, 39%, 40%, 41%, 42%, 43%, 44%, 45%, 46%, 47%, 48%, 49%, 50%, 51%, 52%, 53%, 54%, or 55% by weight of the composition. In some embodiments, the total amount of the one or more diluent(s) in the composition is about 35%, 36%, 37%, 38%, 39%, or 40% to about 41%, 42%, 43%, 44%, 45%, 46%, 47%, 48%, 49%, 50%, 51%, 52%, 53%, 54%, or 55% by weight of the composition. In some embodiments, the diluent makes up the balance of the composition to total 100% by weight of the composition.

One or more (e.g., 1, 2, 3, 4, 5, or more) surfactant(s) may be present in the composition. Example surfactants include, but are not limited to, amphoteric surfactants such as, e.g., cocamidopropyl betaine (e.g., Schercotaine™ CAB-35 betaine commercially available from Lubrizol); anionic surfactants such as, e.g., sodium lauryl sulfoacetate (e.g., Lanthanol® LAL commercially available from Stepan Company); non-ionic surfactants such as, e.g., alkyl polyglucoside (APG®) surfactants such as, e.g., decyl glucoside (e.g., Plantaren® 2000 N UP commercially available from BASF Care Creations); coco-glucoside and/or glyceryl oleate (e.g., Lamesoft® PO 65 commercially available from BASF Care Creations); and any combination thereof. In some embodiments, a surfactant may be present in the composition in an amount of about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition. In some embodiments, a surfactant may be present in the composition in an amount of about 1%, 2%, 3%, 4%, or 5% to about 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition. In some embodiments, the total amount of the one or more surfactant(s) in the composition is about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition. In some embodiments, the total amount of the one or more surfactant(s) in the composition is about 5%, 6%, 7%, 8%, 9%, 10%, 11%, 12%, 13%, 14%, or 15% to about 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, or 50% by weight of the composition.

One or more (e.g., 1, 2, 3, 4, 5, or more) thickening agent(s) may be present in the composition. Example thickening agents include, but are not limited to, acrylates copolymers, such as, e.g., those commercially available from Lubrizol under the tradename Carbopol®, e.g., Carbopol® Aqua SF-1 Polymer. In some embodiments, a thickening agent may be present in the composition in an amount of about 1%, 2%, 3%, 4%, 5%, 6%, 7%, 8%, 9%, 10%, 11%, 12%, 13%, 14%, 15%, 16%, 17%, 18%, 19%, or 20% by weight of the composition. In some embodiments, a thickening agent may be present in the composition in an amount of about 1%, 2%, 3%, 4%, or 5% to about 6%, 7%, 8%, 9%, 10%, 11%, 12%, 13%, 14%, 15%, 16%, 17%, 18%, 19%, or 20% by weight of the composition. In some embodiments, the total amount of the one or more thickening agent(s) in the composition is about 1%, 2%, 3%, 4%, 5%, 6%, 7%, 8%, 9%, 10%, 11%, 12%, 13%, 14%, 15%, 16%, 17%, 18%, 19%, or 20% by weight of the composition. In some embodiments, the total amount of the one or more thickening agent(s) in the composition is about 1%, 2%, 3%, 4%, or 5% to about 6%, 7%, 8%, 9%, 10%, 11%, 12%, 13%, 14%, 15%, 16%, 17%, 18%, 19%, or 20% by weight of the composition.

One or more (e.g., 1, 2, 3, 4, 5, or more) humectant(s) may be present in the composition. Example humectants include, but are not limited to, polyols such as, e.g., glycerin and diols such as, e.g., propanediol (e.g., Zemea® propanediol commercially available from DuPont Tate & Lyle Bio Products Company, LLC); xylitylglucoside, anhydroxylitol, and/or xylitol such as, e.g., commercially available from Seppic under the tradename AQUAXYL™; high hydrophile-lipophile balance (HLB), nonionic emulsifiers and emollients such as, e.g., polyethylene glycol (PEG) 45 palm kernel glycerides (e.g., Crovol™ PK-70 commercially available from Croda, Inc.); sodium hyaluronate (e.g., hyaluronic acid 1%); propylene glycol; polyethylene glycol; polypropylene glycol; triethylene glycol; neopental glycols; butylene glycol; polyethylene glycol; sorbitol; arabitol; erythritol; HSH; isomalt; lactitol; maltitol; mannitol; xylitol; threitol; ribitol; galactitol; fucitol; iditol; inositol; volemitol; and any combination thereof. In some embodiments, a humectant may be present in the composition in an amount of about 0.1%, 0.5%, 1%, 1.5%, 2%, 2.5%, 3%, 3.5%, 4%, 4.5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or 15% by weight of the composition. In some embodiments, a humectant may be present in the composition in an amount of about 0.1%, 0.5%, or 1% to about 1.5%, 2%, 2.5%, 3%, 3.5%, 4%, 4.5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or 15% by weight of the composition. In some embodiments, the total amount of the one or more humectant(s) in the composition is about 0.1%, 0.5%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or 15% by weight of the composition. In some embodiments, the total amount of the one or more humectant(s) in the composition is about 0.1%, 0.5%, 1%, 1.5%, or 2% to about 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, or 15% by weight of the composition.

One or more (e.g., 1, 2, 3, 4, 5, or more) pH adjustment agent(s) may be present in the composition. Example pH adjustment agents include, but are not limited to, bases such as, e.g., sodium hydroxide, potassium hydroxide, and mixtures thereof; acids such as, e.g. hydrochloric acid, citric acid, lactic acid, glycolic acid, acetic acid, and mixtures thereof; sodium carbonate; trolamine; tromethamine; aminomethyl propanol; triisopropanolamine; aminomethyl propanol; tetrahydroxypropyl ethylenediamine; tetrasodium EDTA; suttocide A; triethanolamine (e.g., Trolamine 99); and any combination thereof. In some embodiments, a pH adjustment agent may be present in the composition in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, or 3% by weight of the composition. In some embodiments, a pH adjustment agent may be present in the composition in an amount of about 0.01%, 0.02%, 0.03%, 0.04%, 0.05%, 0.06%, 0.07%, 0.08%, 0.09%, 0.1%, 0.2%, 0.3%, 0.4%, or 0.5% to about 0.6%, 0.7%, 0.8%, 0.9%, 1%, 1.5%, 2%, 2.5%, or 3% by weight of the composition. In some embodiments, the total amount of the one or more pH adjustment agent(s) in the composition is about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, or 3% by weight of the composition. In some embodiments, the total amount of the one or more pH adjustment agent(s) in the composition is about 0.01%, 0.02%, 0.03%, 0.04%, 0.05%, 0.06%, 0.07%, 0.08%, 0.09%, 0.1%, 0.2%, 0.3%, 0.4%, or 0.5% to about 0.6%, 0.7%, 0.8%, 0.9%, 1%, 1.5%, 2%, 2.5%, or 3% by weight of the composition.

One or more (e.g., 1, 2, 3, 4, 5, or more) preservative(s) may be present in the composition. Example preservatives include, but are not limited to, sorbic acid, benzoic acid, methyl-paraben, propyl-paraben, methylchloroisothiazolinone, metholisothiazolinone, diazolidinyl urea, chlorobutanol, triclosan, benzethonium chloride, p-hydroxybenzoate, chlorhexidine, digluconate, hexadecyltrimethyl ammonium bromide, alcohols, benzalkonium chloride, boric acid, bronopol, butylparaben, butylene calcium acetate, calcium chloride, calcium lactate, carbon dioxide, cationic, and bentonite, cetrimide, cetylpyridinium chloride, chlorhexidine, chlorobutanol, chlorocresol, chloroxylenol, citric acid monohydrate, cresol, dimethyl ether, ethylparaben, glycerin, hexetidine, imidurea, isopropyl alcohol, lactic acid, monothioglycerol, pentetic acid, phenol, phenoxyethanol, phenylethyl alcohol, phenylmercuric acetate, phenylmercuric borate, phenylmercuric nitrate, potassium benzoate, potassium metabisulfite, potassium sorbate, propionic acid, propyl gallate, propylene glycol, sodium acetate, sodium benzoate, sodium borate, sodium lactate, sodium sulfite, sodium propionate, sodium metabisulfite, xylitol, sulphur dioxide, carbon dioxide, phenoxyethanol and/or ethylhexylglycerin (e.g., EUXYL® PE 9010 commercially available from Schülke Inc.), and any combination thereof. In some embodiments, a preservative may be present in the composition in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, or 3%, by weight of the composition. In some embodiments, a preservative may be present in the composition in an amount of about 0.01%, 0.02%, 0.03%, 0.04%, 0.05%, 0.06%, 0.07%, 0.08%, 0.09%, 0.1%, 0.2%, 0.3%, 0.4%, or 0.5% to about 0.6%, 0.7%, 0.8%, 0.9%, 1%, 1.5%, 2%, 2.5%, or 3%, by weight of the composition. In some embodiments, the total amount of the one or more preservative(s) in the composition is about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, or 3% by weight of the composition. In some embodiments, the total amount of the one or more preservative(s) in the composition is about 0.01%, 0.02%, 0.03%, 0.04%, 0.05%, 0.06%, 0.07%, 0.08%, 0.09%, 0.1%, 0.2%, 0.3%, 0.4%, or 0.5% to about 0.6%, 0.7%, 0.8%, 0.9%, 1%, 1.5%, 2%, 2.5%, or 3% by weight of the composition.

One or more (e.g., 1, 2, 3, 4, 5, or more) cooling agent(s) may be present in the composition. Example cooling agents include, but are not limited to, menthyl ethylamido oxalate (e.g., Frescolat® X-cool commercially available from Symrise). In some embodiments, a cooling agent may be present in the composition in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, or 3% by weight of the composition. In some embodiments, a cooling agent may be present in the composition in an amount of about 0.01%, 0.02%, 0.03%, 0.04%, 0.05%, 0.06%, 0.07%, 0.08%, 0.09%, 0.1%, or 0.2% to about 0.3%, 0.4%, 0.5%, 0.6%, 0.7%, 0.8%, 0.9%, 1%, 1.5%, 2%, 2.5%, or 3% by weight of the composition. In some embodiments, the total amount of the one or more cooling agent(s) in the composition is about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, or 3% by weight of the composition. In some embodiments, the total amount of the one or more cooling agent(s) in the composition is about 0.01%, 0.02%, 0.03%, 0.04%, 0.05%, 0.06%, 0.07%, 0.08%, 0.09%, 0.1%, or 0.2% to about 0.3%, 0.4%, 0.5%, 0.6%, 0.7%, 0.8%, 0.9%, 1%, 1.5%, 2%, 2.5%, or 3% by weight of the composition.

One or more (e.g., 1, 2, 3, 4, 5, or more) chelating agent(s) may be present in the composition. Example chelating agents include, but are not limited to, tetrasodium salt of ethylenediaminetetraacetate (EDTA). (e.g., VERSENE™ 100 XL commercially available from The Dow Chemical Company); disodium salt of EDTA (e.g., VERSENE™ NA commercially available from The Dow Chemical Company); and combinations thereof. In some embodiments, a chelating agent may be present in the composition in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, or 3% by weight of the composition. In some embodiments, a chelating agent may be present in the composition in an amount of about 0.01%, 0.02%, 0.03%, 0.04%, 0.05%, 0.06%, 0.07%, 0.08%, or 0.09% to about 0.1%, 0.2%, 0.3%, 0.4%, 0.5%, 0.6%, 0.7%, 0.8%, 0.9%, 1%, 1.5%, 2%, 2.5%, or 3% by weight of the composition. In some embodiments, the total amount of the one or more chelating agent(s) in the composition is about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, or 3% by weight of the composition. In some embodiments, the total amount of the one or more chelating agent(s) in the composition is about 0.01%, 0.02%, 0.03%, 0.04%, 0.05%, 0.06%, 0.07%, 0.08%, or 0.09% to about 0.1%, 0.2%, 0.3%, 0.4%, 0.5%, 0.6%, 0.7%, 0.8%, 0.9%, 1%, 1.5%, 2%, 2.5%, or 3% by weight of the composition.

L-arginine and/or L-citrulline and/or esters and/or derivatives thereof may be present in a composition of the present invention. In some embodiments, L-arginine and/or L-citrulline and/or esters and/or derivatives thereof may be present in the composition in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, or 5% by weight of the composition. In some embodiments, L-arginine and/or L-citrulline and/or esters and/or derivatives thereof may be present in the composition in an amount of about 0.01%, 0.02%, 0.03%, 0.04%, 0.05%, 0.06%, 0.07%, 0.08%, 0.09%, 0.1%, 0.2%, 0.3%, 0.4%, 0.5%, 0.6%, 0.7%, 0.8%, 0.9%, or 1% to about 1.5%, 2%, 2.5%, 3%, 3.5%, 4%, 4.5%, or 5% by weight of the composition.

In some embodiments, L-arginine and L-citrulline and/or esters and/or derivatives thereof may be present in a composition of the present invention in a ratio of about 3:1 to about 1:3 (i.e., L-arginine and/or esters and/or derivatives thereof to L-citrulline and/or esters and/or derivatives thereof), such as, e.g., about 3:1, 2:1, 1:1:, 1:2, or 1:3. In some embodiments, L-arginine and L-citrulline and/or esters and/or derivatives thereof may be present in a composition of the present invention in a ratio of about 1:1.

Example compositions of the present invention include, but are not limited to, those provided in Table 1.

TABLE 1
Example compositions of the present invention.

| Ingredient (INCI Name) | Trade Name | Function | % w/w |
|---|---|---|---|
| Purified Water | Purified Water | Diluent | 35%-balance |
| | | | (e.g., 35%-50%) |
| Cocamidopropyl Betaine | Schercotaine CAB-35 | Surfactant | 10%-20% |
| Sodium Lauryl Sulfoacetate | Lanthanol LAL | Surfactant | 5%-15% |
| Decyl Glucoside | Plantaren 2000N UP | Surfactant | 5%-15% |
| Acrylates Copolymer | Carbopol Aqua SF-1 | Thickener | 1%-15% |
| Coco-Glucoside (and) | Lamesoft PO 65 | Surfactant | 0.1%-10% |
| Glyceryl Oleate | | | |
| Propanediol | Zemea Propanediol | Humectant/Emollient | 0.1%-5% |
| Glycerin | Glycerin | Humectant/Emollient | 0.1%-5% |
| Xylitylglucoside (and) | Aquaxyl | Humectant/Emollient | 0.1%-5% |
| Anhydroxylitol (and) | | | |
| Xylitol | | | |
| Triethanolamine | Trolamine 99 | pH Adjustment | 0.01%-2% |
| Phenoxyethanol (and) | Euxyl PE 9010 | Preservative | 0.01%-2% |
| Ethylhexylglycerin | | | |
| PEG 45 Palm Kernel | Crovol PK 70 | Humectant/Emollient | 0.01%-2% |
| Glycerides | | | |
| Sodium Hyaluronate | Hyaluronic Acid 1% | Humectant/Emollient | 0.01%-2% |
| Menthyl Ethylamido | Frescolat X-Cool | Cooling Agent | 0.01%-2% |
| Oxalate | | | |
| Tetrasodium EDTA | Versene 100 XL | Chelating Agent | 0.01%-2% |

| | |
|---|---|
| To Make Total | 100.0% |

A composition of the present invention may comprise an active pharmaceutical ingredient (API). Any suitable API or combinations of APIs may be included in a composition of the present invention. Example APIs include, but are not limited to, antimicrobial agents, anti-acne agents, anti-inflammatory agents, analgesic agents, anesthetic agents, antihistamine agents, antiseptic agents, immunosuppressants, antihemorrhagic agents, vasodilators, wound healing agents, anti-biofilm agents, and any combination thereof. Example APIs include, but are not limited to, those described in International Application Publication No. WO 2013/006608, which is incorporated herein by reference in its entirety. In some embodiments, a composition of the present invention may not comprise an API.

In some embodiments, the composition comprises an API and the API is an anti-acne agent. One or more (e.g., 1, 2, 3, 4, 5, or more) anti-acne agents may be present in the composition. In some embodiments, the API is salicylic acid. Salicylic acid may be about present in the composition in an amount of about 0.1%, 0.5%, 1%, 2%, 3%, 4%, 5%, 6%, 7%, 8%, 9%, or 10% by weight of the composition. In some embodiments, salicylic acid is present in the composition in an amount of about 0.1%, 0.5%, 1%, or 1.5% to about 2%, 2.5%, 3%, 3.5%, 4%, 4.5%, or 5% by weight of the composition.

A composition of the present invention may have a pH in a range of about 4 to about 8. In some embodiments, the composition may have a pH of about 4, 4.5, 5, 5.5, 6, 6.5, 7,7.5, or 8.

A composition of the present invention may have a viscosity in a range of about 5,000 cP to about 25,000 cP, such as, but not limited to, about 5,000 cP to about 20,000 cP or about 7,000 cP to about 15,000 cP. In some embodiments, a composition of the present invention may have a viscosity of about 5,000, 5,500, 6,000, 6,500, 7,000, 7,500, 8,000, 8,500, 9,000, 9,500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, 20,000, 20,500, 21,000, 21,500, 22,000, 22,500, 23,000, 23,500, 24,000, 24,500, or 25,000 cP.

In some embodiments, a kit is provided. The kit comprises a composition of the present invention and optionally one or more (e.g., 1, 2, 3, or more) different compositions. In some embodiments, a kit of the present invention comprising two or more (e.g., 2, 3, 4, 5, 6, 7, or more) separately stored compositions of the present invention. For example, each of the two or more separately stored composition may be in individual containers such as, e.g., vials, pouches, sachets, and/or the like. A kit of the present invention may provide a 7, 14, 21, or 30 day supply of the composition of the present invention.

According to some embodiments, a method of the present invention may comprise administering a composition of the present invention to the skin of a subject. In some embodiments, the composition may be topically administered.

In some embodiments, a method of the present invention comprises delivering a therapeutically effective amount of a composition of the present invention to the skin of a subject. As used herein, the term “therapeutically effective amount” refers to an amount of a composition of the present invention that elicits a therapeutically useful response in a subject. Those skilled in the art will appreciate that the therapeutic effects need not be complete or curative, as long as some benefit is provided to the subject. In some embodiments, a therapeutically effective amount of a composition of the present invention may include delivering a therapeutically effective amount of a component of the composition, such as, but not limited to, an active pharmaceutical ingredient.

The present invention finds use in both veterinary and medical applications. Subjects suitable to be treated with a method embodiment of the invention include, but are not limited to, avian and mammalian subjects. Mammals of the present invention include, but are not limited to, canines, felines, bovines, caprines, equines, ovines, porcines, rodents (e.g. rats and mice), lagomorphs, primates (e.g., simians and humans), non-human primates (e.g., monkeys, baboons, chimpanzees, gorillas), and the like, and mammals in utero. Any mammalian subject in need of being treated according to the present invention is suitable. Human subjects of both genders and at any stage of development (i.e., neonate, infant, juvenile, adolescent, adult) may be treated according to the present invention. In some embodiments of the present invention, the subject is a mammal and in certain embodiments the subject is a human. Human subjects include both males and females of all ages including fetal, neonatal, infant, juvenile, adolescent, adult, and geriatric subjects as well as pregnant subjects. In particular embodiments of the present invention, the subject is a human adolescent and/or adult.

Illustrative avians according to the present invention include chickens, ducks, turkeys, geese, quail, pheasant, ratites (e.g., ostrich) and domesticated birds (e.g., parrots and canaries), and birds in ovo.

The methods of the present invention may also be carried out on animal subjects, particularly mammalian subjects such as mice, rats, dogs, cats, livestock and horses for veterinary purposes and/or for drug screening and drug development purposes.

In some embodiments of the present invention, the subject is “in need of” a method of the present invention, e.g., the subject has been diagnosed with, is at risk for, and/or is believed to have a disease or disorder that may be treated using a method of the present invention. In some embodiments, the subject has a skin disorder, such as, but not limited to, acne, androgenetic alopecia, atopic dermatitis, seborrheic dermatitis, tinea infections, candida infections, bacterial infections, verruca vulgaris, and/or psoriasis. In some embodiments of the present invention, the subject has an inflammatory skin condition or disorder and/or infection (e.g., impetigo, leishmaniasis, etc.). In some embodiments, a composition of the present invention may be used to treat acne vulgaris. According to some embodiments, a composition and/or method of the present invention may reduce P. acnes counts, inflammatory lesions, and/or noninflammatory lesions in a subject.

“Treat,” “treating” or “treatment of” (and grammatical variations thereof) as used herein refer to any type of treatment that imparts a benefit to a subject and may mean that the severity of the subject's condition is reduced, at least partially improved or ameliorated and/or that some alleviation, mitigation or decrease in at least one clinical symptom is achieved and/or there is a delay in the progression of the disease, disorder, and/or condition. In particular embodiments, the severity of a skin disorder (e.g., acne) may be reduced in a subject compared to the severity of the skin disorder in the absence of a method of the present invention. In other embodiments, a method of the present invention may prevent and/or treat against infection.

A composition of the present invention may be applied topically to any portion of a subject's skin. However, in some embodiments, the subject's face is treated by a method described herein. Furthermore, in some embodiments, the subject's trunk is treated by a method described herein. In some embodiments, the subject's back, arm(s), hand(s), finger(s), foot, feet, toe(s), and/or genital(s) are treated by a method described herein.

According to some embodiments of the present invention, a method of treating acne vulgaris may be provided, the method comprising topically applying a composition of the present invention to the skin of a subject. A therapeutically effective amount of the composition may be applied.

According to further embodiments of the present invention, a method of reducing inflammatory and/or noninflammatory lesions in a subject may be provided comprising topically applying a composition of the present invention to the skin of the subject. A therapeutically effective amount of the composition may be applied. In some embodiments, the method may reduce inflammatory and/or noninflammatory lesions by about 10% or greater, such as, but not limited to, about 15%, 20%, 25%, 30%, 35%, 40%, 45%, 50%, 55%, 60%, 65%, 70%, 75%, 80%, 85%, 90%, 95%, or more, over a defined period of time compared to a subject who did not apply a composition of the present invention over the same time period. In some embodiments, the subject may see a reduction in inflammatory and/or noninflammatory lesions within about 1, 2, 3, 4, 5, 6, 7, 8, 9, 10, 11, 12, 13, or more week(s). In some embodiments, the method may reduce inflammatory and/or noninflammatory lesions in the skin of the subject within 12 weeks or less, in some embodiments, within 8 weeks or less, and in further embodiments, within 4 weeks or less.

In some embodiments, a method of the present invention may reduce P. acnes counts in the subject. In some embodiments, a method of the present invention may reduce P. acnes counts by about 10% or greater, such as, but not limited to, about 15%, 20%, 25%, 30%, 35%, 40%, 45%, 50%, 55%, 60%, 65%, 70%, 75%, 80%, 85%, 90%, 95%, or more over a defined period of time compared to a subject who did not apply a composition of the present invention over the same time period. In some embodiments, a reduction in P. acnes counts may occur within about 1, 2, 3, 4, 5, 6, 7, 8, 9, 10, 11, 12, 13, or more week(s). In some embodiments, a method of the present invention may reduce P. acnes counts in the skin of the subject within 12 weeks or less, in some embodiments, within 8 weeks or less, and in further embodiments, within 4 weeks or less.

The foregoing is illustrative of the present invention, and is not to be construed as limiting thereof. The invention is defined by the following claims, with equivalents of the claims to be included therein. All publications, patent applications, patents, patent publications, and other references cited herein are incorporated by reference in their entireties for the teachings relevant to the sentence and/or paragraph in which the reference is presented.

That which is claimed is:

1. A composition comprising:
a diluent,
a surfactant,
a thickening agent,
a humectant,
a cooling agent,
a chelating agent,
optionally a pH adjustment agent, and
optionally a preservative.

2. The composition of claim 1, further comprising L-arginine and/or L-citrulline and/or esters and/or derivatives thereof, optionally wherein L-arginine and/or L-citrulline and/or esters and/or derivatives thereof is present in an amount of about 0.01% to about 5% by weight of the composition.

3. The composition of any preceding claim, wherein the diluent is water.

4. The composition of any preceding claim, wherein the diluent is present in an amount of about 35% to about 50% by weight of the composition.

5. The composition of any preceding claim, wherein the surfactant is selected from cocamidopropyl betaine, sodium lauryl sulfoacetate, decyl glucoside, coco-glucoside and/or glyceryl oleate, and any combination thereof.

6. The composition of any preceding claim, wherein the surfactant is present in an amount of about 5% to about 50% by weight of the composition.

7. The composition of any preceding claim, wherein the thickening agent is an acrylates copolymer.

8. The composition of any preceding claim, wherein the thickening agent is present in an amount of about 3% about 20% by weight of the composition.

9. The composition of any preceding claim, wherein the humectant is selected from propanediol, glycerin, xylitylglucoside, anhydroxylitol, xylitol, PEG 45 palm kernel glycerides, sodium hyaluronate, and any combination thereof, optionally wherein the humectant is selected from propanediol; glycerin; xylitylglucoside, anhydroxylitol, and/or xylitol; PEG 45 palm kernel glycerides; sodium hyaluronate; and any combination thereof.

10. The composition of any preceding claim, wherein the humectant is present in an amount of about 0.1% about 15% by weight of the composition.

11. The composition of any preceding claim, wherein the cooling agent is menthyl ethylamido oxalate.

12. The composition of any preceding claim, wherein the cooling agent is present in an amount of about 0.01% about 2% by weight of the composition.

13. The composition of any preceding claim, wherein the chelating agent comprises EDTA, optionally wherein the chelating agent is tetrasodium EDTA.

14. The composition of any preceding claim, wherein the chelating agent is present in an amount of about 0.01% about 2% by weight of the composition.

15. The composition of any preceding claim, wherein the composition comprises the pH adjustment agent and the pH adjustment agent is triethanolamine.

16. The composition of any preceding claim, wherein the composition comprises the pH adjustment agent and the pH adjustment agent is present in an amount of about 0.01% to about 3% by weight of the composition.

17. The composition of any preceding claim, wherein the composition comprises the preservative and the preservative comprises phenoxyethanol and/or ethylhexylglycerin.

18. The composition of any preceding claim, wherein the composition comprises the preservative and the preservative is present in an amount of about 0.01% to about 3% by weight of the composition.

19. The composition of any preceding claim, further comprising an active pharmaceutical ingredient (e.g., salicylic acid).

20. The composition of any preceding claim, wherein the composition is cosmetically elegant.

21. A kit comprising a composition of any preceding claim.

22. The kit of claim 21, further comprising a different separately stored composition.

22. The kit of claim 21, wherein the composition is separately stored in two or more containers.

23. A method of cleaning the skin of a subject, the method comprising applying a composition of any one of claims 1-20 to the skin of the subject, thereby cleaning the skin of the subject.

24. A method of treating a subject, the method comprising applying a composition of any one of claims 1-20 to the skin of the subject, thereby treating the subject.

25. A compound, composition, article of manufacture and/or method substantially as shown and/or described herein.
